(19) United States
(12) Patent Application Publication
(10) Pub. No.: US 2006/0141473 A1
(43) Pub. Date: Jun. 29, 2006

(54) Erk7 and erk8, novel diagnostic markers for cancer

(76) Inventors: Deborah A. Lannigan-Macara, Charlottesville, VA (US); Lorin M. Henrich, Waynesboro, VA (US); Jeffrey A. Smith, Earlysville, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 10/560,470

(22) PCT Filed: Jun. 17, 2004
(86) PCT No.: PCT/US04/19181

§ 371 Date: Dec. 14, 2005

Related U.S. Application Data
(60) Provisional application No. 60/478,992, filed on Jun. 17, 2003.

(51) Int. Cl. C12Q 1/68; G01N 33/574

(52) U.S. Cl. 435/6; 435/7.23

(57) ABSTRACT

The present invention is directed in part to the discovery of a novel signal transduction pathway that regulates estrogen responsiveness. Human extracellular signal-regulated kinase 8 (ERK8) has been discovered by applicants to preferentially enhance the destruction of ERα, and loss of ERK8 is correlated with breast cancer progression. Thus monitoring the expression of ERK8 can be used as a diagnostic and therapeutic indicator of cancer and cancer progression.

### BACKGROUND

Estrogen receptor alpha (ERα) belongs to the superfamily of ligand-activated transcription factors. This superfamily shares a common modular structure, which consists of an N-terminal region, a DNA-binding domain and a ligand-binding domain. ERα regulates the expression of genes involved in growth and development. The cellular response to estrogens in vivo is ERα-limited and a key mechanism in regulating ERα concentration is receptor degradation. In response to estradiol, the rate of ERα degradation through ubiquitination and the 26S proteasome pathway is increased by an unknown mechanism. The 26S proteasome pathway is the major pathway of regulated proteolysis in eukaryotes and is responsible for the destruction of ubiquitinated substrates.

Parallel studies on other members of the nuclear receptor superfamily have suggested a role for mitogen-activated protein kinase (MAPK(also referred to as extracellular signal regulated kinase (ERK)1/2)) in regulating receptor turnover. For example, MAPK(ERK1/2) phosphorylation of Ser-294 in the human progesterone receptor (PR) increases the PR degradation rate. MAPK(ERK1/2) is also known to phosphorylate ERα, and therefore whether MAPK(ERK1/2) enhances ERα turnover was investigated.

MAPK(ERK1/2) belongs to a kinase subgroup the activity of which is regulated by phosphorylation/dephosphorylation of a threonine and tyrosine residue present in a Thr-Glu-Tyr (TEY) motif within the activation loop. Other members of this subgroup include ERK5, ERK7 and ERK8. ERK8 is the human functional homolog of rodent ERK7. ERK7 and ERK8 (ERK7/8) differs from the other members of the TEY subgroup in multiple ways. At least 50% of ectopically expressed ERK7/8 is active in growth factor-free conditions, while MAPK(ERK1/2) and ERK5 require growth factor stimulation for activation. Furthermore, MAPK(ERK1/2) must be co-expressed with a constitutively active MAPK kinase(MEK) in order for phosphorylation of the TEY motif in bacterial expression systems. In contrast, the TEY motif of ERK7/8 is substantially phosphorylated in bacteria, suggesting that primary mechanism of ERK7/8 activation is by autophosphorylation. In addition, treatment of cells expressing MAPK(ERK1/2) with okadaic acid, a protein phosphatase 2A (PP2A) inhibitor, increases the amount of TEY phosphorylated MAPK(ERK1/2). However, okadaic acid has no effect on ERK7/8 TEY phosphorylation. Therefore, both activation and inactivation of ERK7/8 are distinct from other members of the TEY subgroup.

The substrate specificity of ERK7/8 differs from that of MAPK(ERK1/2). MAPK(ERK1/2) is able to phosphorylate a variety of substrates including c-Jun and Elk-1 in-vitro. However, ERK7/8 is unable to phosphorylate these substrates. The cellular localization of ERK7/8 is not regulated in the same manner as MAPK(ERK1/2). MAPK(ERK1/2) and ERK5 require growth factor activation for translocation to the nucleus, while in overexpression experiments ERK7/8 appears to be constitutively nuclear. ERK7/8 also possesses a unique C-terminal tail, which is absent in MAPK(ERK1/2) and shares no similarity with the C-terminal tail of ERK5. The C-terminal tail is required for nuclear localization of ERK7/8 and may contain regions important for protein-protein interactions. In summary, ERK7/8 is unique from other TEY family members in regulation of activation, substrate specificity, and nuclear localization.

ERK7/8 differs substantially from not only MAPK family members, but also all other known kinases. A unique identifier of ERK7/8 is the presence of a glutamine (Q) at position 139 in subdomain VIB of the kinase domain in ERK7 (position 138 in ERK8). This domain is essential for the catalytic activity of kinases. A search of the Protein Kinase Database revealed that there are no other kinases in the human kinome that contain a polar residue at this position. Molecular modeling of the equivalent residue in the crystal structure of ERK2 indicates that a glutamine at this position may form three hydrogen bonds with residues in the catalytic domain of ERK7/8. One of the predicted residues that may become hydrogen bonded is aspartate 138 in ERK7 (aspartate 137 in ERK8), a residue responsible for co-ordination of the substrate for phosphotransfer. Thus Q139 in ERK7 (Q138 in ERK8) may significantly alter the biological properties of ERK 7/8. In in-vitro studies using myelin basic protein as a substrate, autophosphorylated ERK7/8 is approximately 1000× less active than activated MAPK(ERK1/2). It is possible that Q139 may play a role in limiting kinase activity of ERK7/8. Interestingly, mutation of Q139 to leucine, a hydrophobic residue, enhances ERK7/8 TEY phosphorylation in cell-based studies.

ERα is believed to play a key role during the development of breast and endometrial cancers. It has been reported that an up-regulation of expression levels of ERα occurs during the development of intraductal carcinomas from normal mammary glands, and a decrease in their expression levels occurs during the progression of breast cancer. More particularly, loss of ERα is associated with aggressive breast tumors and poor clinical outcome.

Very little is known about ERK7, and its biological function has not been established. As reported herein ERK7/8 preferentially enhances the destruction of ERα but not the related androgen receptor. Other protein kinases closely related to ERK7/8 do not enhance ERα turnover, and ERK7 kinase activity is required for its effect on ERα. In human breast cells, a dominant-negative ERK7 mutant decreased the rate of endogenous ERα degradation >4-fold in the presence of hormone, and potentiated estrogen responsiveness. ERK7 targets the ERα ligand-binding domain for destruction by enhancing its ubiquitination. As described herein, loss of ERK7/8 has now been correlated with breast cancer progression, and all ERα-positive breast tumors tested had decreased ERK7/8 expression compared to normal breast tissue.

As reported herein, the present studies have revealed the existence of a new signaling pathway impinging on the 26S proteasome machinery, in which ERK7/8 regulates hormone responsiveness in breast cells by controlling the rate of ERα degradation. Furthermore, the loss of this pathway appears to be correlated to the development of breast cancer.

### SUMMARY OF VARIOUS EMBODIMENTS

In accordance with one embodiment of the invention a method of diagnosing the presence of an estrogen responsive cancer in a patient and determining a prognosis for the patient is provided. The method comprises the step of measuring ERK8 levels in the cells of a biological sample obtained from said patient (i.e. a biopsy), and determining if the ERK8 levels of the biological sample are significantly lower than those detected in non-cancerous cells (either from that patient or from population data), wherein significantly lower ERK8 levels indicates the presence of cancer cells in said patient.

### BRIEF DESCRIPTION OF THE DRAWINGS

FIG. 1: is a graph representing the determination of the K_{M} of ATP for bacterially expressed ERK7. Purified ERK7 (5 nM) was incubated in the presence of increasing ATP concentrations. The concentration of ATP supporting maximal velocity of the kinase reaction is 1 mM. The concentration of ATP supporting half-maximal velocity (K_{M}) is ˜100 μM. Background is the measured signal from reactions carried out in the presence of 200 mM EDTA, which eliminates kinase activity. The background has not been subtracted from the ERK7 signal. Phosphorylation of the immobilized GST-MBPtide was detected using the HRP-ELISA as described in Example 1.

FIG. 2: is a bar graph representing the activity of recombinant ERK7 and ERK8 and immunoprecipitated ERK7 as measured by phosphorylation of GST-MBPtide. Purified, bacterially-expressed ERK7 and ERK8 (5 nM) and ERK7 immunoprecipitated from approximately 10% of a confluent 15 cm tissue culture plate was incubated in the presence of 1 mM ATP. The background values determined in the presence of 200 mM EDTA have been subtracted. Phosphorylation of the immobilized GST-MBPtide was detected using the HRP-ELISA as described in Example 1.

FIG. 3: demonstrates the alignment of rat ERK7 (SEQ ID NO: 2), mouse ERK7 (SEQ ID NO: 3) and human ERK8 (SEQ ID NO: 1). GenBank sequences for rat ERK7(Acc# AF078798), mouse ERK7(Acc# BC48042), and human ERK8(Acc# AY065978) were aligned using EMBL-EBI Clustal W multiple sequence alignment program at http://www.ebi.ac.uk/clustalw/. Results are presented as a similarity chart where “*” represents identical residues, “:” represents highly similar residues, “.” represents less similar residues, and gaps represent no similarity.

FIG. 4: is a bar graph representing the expression levels of ERK8 in various breast cancers wherein the data has been normalized to the level observed in normal breast tissue.

FIG. 5 is a bar graph representing the expression levels of ERK8 in various breast cancers in which the grade of tumor was clearly known.

FIG. 6 represents a photograph of a Western blot demonstrating that the anti-ERK7 antibody recognizes recombinant human ERK8. Recombinant human ERK8 containing an N-terminal MYC tag and a C-terminal His tag was purified from bacteria, electrophoresed, and immunoblotted with the indicated antibody. The pTEY antibody is an antibody specific to the dually-phosphorylated TEY motif The lower molecular weight band present in the anti-pTEY blot is a degradation product.

FIG. 7 represents a photograph of a Western blot demonstrating that short interfering RNA reduces the detectable signal generated by the anti-ERK7 antibody. Human MCF10A cells, which contain endogenous ERK8, were transfected with control short-interfering(si) RNA, ERK8-specific siRNA, or RSK2-specific siRNA. The cells were serum-starved for 24 hrs and then lysed in boiling sample buffer. Lysates containing equal amounts of total protein were electrophoresed and immunoblotted with the indicated antibody. Detection of equivalent amounts of total ERK1/2, recognized by the anti-pan ERK antibody, indicates that the samples were correctly normalized.

FIG. 8 is a bar graph representing data demonstrating that active ERK7 enhances ERα destruction. BHK cells were co-transfected with vectors encoding ERα and either the indicated kinase (ERK7 or K43A-ERK7) or vector control (V). The transfected cells were serum-starved and treated with +/−10 nM estradiol (E2) for 6 hr before the addition of boiling sample buffer. Equal amounts of total protein were electrophoresed and immunoblotted and the relative amounts of ERα were determined from the immunoblots by densitometry. The data are expressed as the % of ERα divided by ERα in the vector control in the absence of E2. The means±standard error (S.E.) are shown for n=8. *P<0.05 and **P<0.005 (Student's t-test) obtained by comparing ERα levels with co-expressed ERK7 to those obtained with the appropriate vector control.

FIGS. 9A & 9B represent Western blots demonstrating that ERK7 specifically enhances the degradation of ERα. BHK cells were co-transfected with vectors encoding HA-SF1 and HA-K43A-ERK7 or HA-ERK7 or vector control (V). The serum-starved, transfected cells were lysed, and equal amounts of protein electrophoresed and immunoblotted, with the results being shown in FIG. 9A. BHK cells were also co-transfected with vectors encoding ERα or HA-AR and HA-ERK7 or MYC-ERK7 or vector control (V) and the transfected cells were then aliquoted and pre-treated with 50 mM cycloheximide for 2 h before the addition of 10 nM E2 or 100 nM dihydroxytestosterone (DHT). Thereafter, at various times after ligand addition the cells were lysed and immunoblotted, with the results being shown in FIG. 9B.

FIG. 10A & 10B represent Western blots demonstrating ERK expression in human breast cells. FIG. 10A represents equivalent amounts of lysate from serum-starved MCF-10A, MCF-7 and MDA-MB-231 cells electrophoresed, immunoblotted and probed with the indicated labeled antibodies. FIG. 10B represents the amount of ERK7 and ERα present in normal human breast tissue, benign tumor tissue and breast cancer tissue. The various tissue samples were solubilized, normalized for Ran expression, electrophoresed and immunoblotted. The tumor grade was obtained from the athologist's report. ERα positive samples are indicated by an *.

FIG. 11 is a graph demonstrating the effects of K43A-ERK7 on ERα-mediated proliferation growth in the presence of vehicle (□) or ICI 182,780 (o), a pure estrogen antagonist. MCF-7 stably transfected with K43A-ERK7 were treated +/−1 mM ICI 182,780. At various times the cells were lysed and growth determined. ERα-dependent growth was obtained by subtracting the growth in ICI 182,780 from the growth obtained in vehicle control. The results are taken from two experiments in which each time point was determined in triplicate and from two independent lines. *P<0.05 (Student's t-test) obtained by comparing the response obtained with the stables expressing HA-K43A-ERK7 to the appropriately treated control.

### DETAILED DESCRIPTION OF EMBODIMENTS

Definitions

In describing and claiming the invention, the following terminology will be used in accordance with the definitions set forth below.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells.

As used herein, the term “linkage” refers to the connection between two groups. The connection can be either covalent or non-covalent, including but not limited to ionic bonds, hydrogen bonding and hydrophobic/hydrophilic interactions.

A “detectable marker” is an atom or molecule that permits the specific detection of a molecule comprising the marker in the presence of similar molecules without a marker. Markers include, for example colored beads, radioactive isotopes, antigenic determinants, enzymes, nucleic acids available for hybridization, chromophors, fluorophors, chemiluminescent molecules, electrochemically detectable molecules and molecules that provide for altered fluorescence-polarization or altered light-scattering.

As used herein the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

As used herein the term “magnetic particles” refers to particles that are responsive to a magnetic field.

As used herein, the term “antibody” refers to a polyclonal or monoclonal antibody or a binding fragment thereof such as Fab, F(ab′)2 and Fv fragments.

As used herein, the term “ERK7/8” refers to generically to either the human, rat or mouse extracellular signal-regulated kinases of SEQ ID NO: 1, SEQ ID NO: 2 and SEQ ID NO: 3, respectively, and fragments thereof.

As used herein, the term “ERK8” refers to the human extracellular signal-regulated kinase 8 polypeptide of SEQ ID NO: 1 and fragments thereof.

As used herein, “ERK7/8 levels” is a generic term that refers to either the concentration of ERK7/8 protein or RNA, or ERK7/8 kinase activity, or any combination thereof.

As used herein, the term “ERK8 antibody” refers to an antibody that specifically binds to the amino acid sequence of SEQ ID NO: 1.

As used herein, “biological sample” means any biological tissue, fluid, serum, or biopsy sample taken from an individual.

An “estrogen responsive” cancer or neoplastic cell is a cell that will increase cellular transcription, metabolism, growth or proliferation upon exposure to estrogen.

The term “neoplastic cells” as used herein refers to cells that result from abnormal new growth. A neoplastic cell may be malignant or benign and includes both blood cancers and solid tumor cells.

As used herein, the term “tumor” refers to an abnormal mass or population of cells that result from excessive cell division, whether malignant or benign, and all pre-cancerous and cancerous cells and tissues. A “tumor” is further defined as two or more neoplastic cells.

### EMBODIMENTS

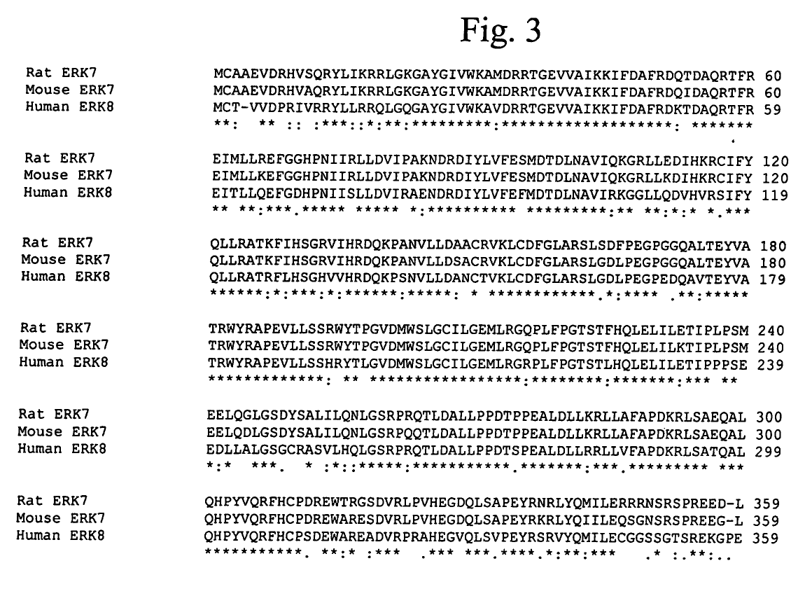

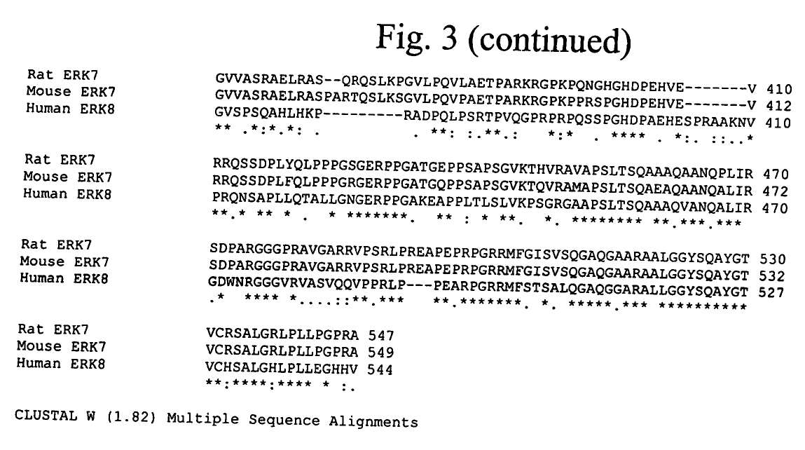

The present invention is directed in part to the discovery of a novel signal transduction pathway that regulates estrogen responsiveness. Human extracellular signal-regulated kinase 8 (ERK8; see SEQ ID NO: 1) was identified based on the ability of two different antibodies to recognize a protein of a MW similar to that predicted for rat ERK7 (SEQ ID NO: 2). Accordingly, ERK8 has been identified as the human functional equivalent of the mouse and rat ERK7 polypeptides. A comparison of the mouse and rat ERK7 and human ERK8 sequences is provided in FIG. 3 and reveals a high degree of sequence similarity between the three polypeptides. As reported herein ERK7/8 has been discovered to preferentially enhance the destruction of ERα but not the related androgen receptor. Furthermore, other protein kinases closely related to ERK7/8 do not enhance ERα turnover, and ERK7/8 kinase activity is required for its effect on ERα.

Further support for the physiological importance of ERK7/8 is provided by applicants' observations that ERK8 is expressed in human breast tissue, an estrogen target, and that there is a decrease in ERK8 expression during breast cancer progression. Furthermore, in all tumor samples that are ERα positive the expression of ERK8 has been lost. This inverse correlation between ERα and ERK8 expression levels was also observed in human breast cell lines.

In addition it is noted that a dominant negative ERK7 mutant is able to regulate hormone responsiveness in breast cells as measured by transcription and proliferation. A dominant negative ERK7 mutant was able to enhance ERα-mediated transcription to the same extent as treatment of wild type tissues with the proteasome inhibitor, MG132, which argues that endogenous ERK7 is the predominant pathway of ERα degradation in breast cells. These results (see Example 1 for details) support a model in which ERK7/8 enhances hormone-dependent ERα destruction through the 26S proteasome pathway by increasing ERα ubiquitination. This model is supported by observations that a dominant negative ERK7 mutant preferentially inhibits degradation of the hormone-bound compared to unbound ERα. ERK7 enhances the level of ubiquitination of the ligand-binding domain and this domain is required for ERK7/8-mediated destruction. In response to hormone binding it is known that the ligand-binding domain undergoes a conformational change and presumably this conformational change targets hormone-bound ERα for destruction by ERK7/8.

It has been previously reported that, in response to hormone, the phosphorylation of Ser-118 increases. ERK7/8 enhances Ser-118 phosphorylation in vivo but this phosphorylation is not important for ERK7/8-mediated degradation because ERK7/8 is able to enhance the rate of degradation of ERα and S118-ERα to the same extent. No evidence has been reported that mutation of other putative ERK7/8 phosphorylation sites influenced ERK7/8-mediated ERα degradation. It seems unlikely that phosphorylation of the ligand-binding domain plays a role in regulating turnover because no enhanced phosphorylation in the ligand-binding domain was observed using (³²P)-orthophosphate labeling and microsequencing of the radiolabeled ERα peptides in the absence or presence of lactacystin. Accordingly, applicants believe that ERK7/8 is important in maintaining the homeostasis of a normal breast cell and that with high frequency ERK7/8 is lost at an early step in breast cancer progression. Perhaps during tumor progression ERK7/8 downstream effectors have been inappropriately activated, resulting in reduced ERα levels even in the absence of ERK7/8.

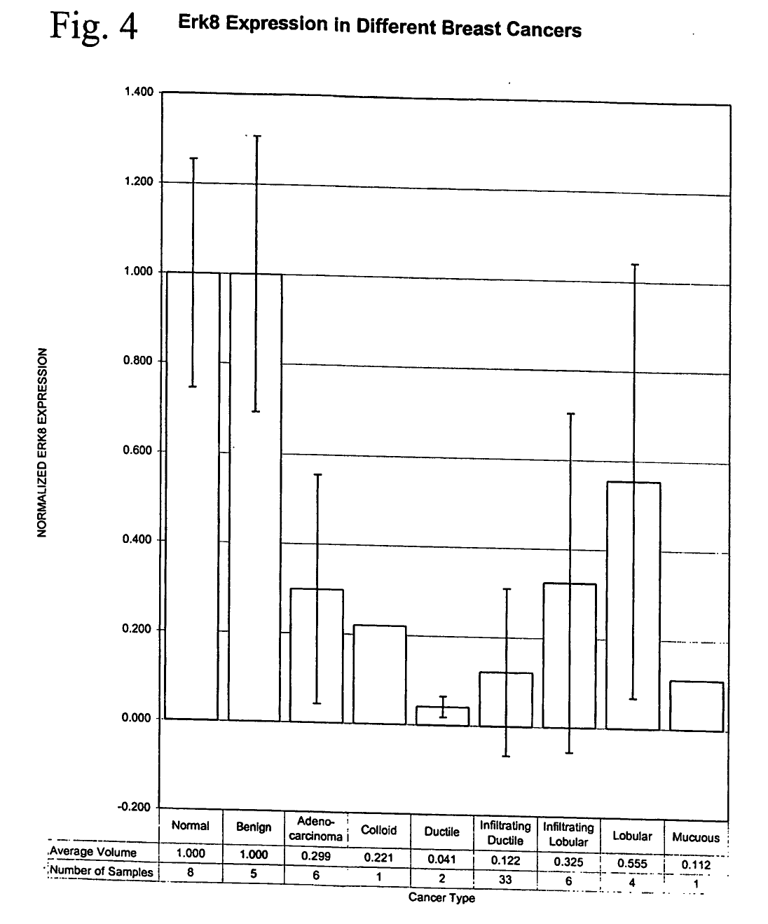

Since the loss of ERK7/8 has been discovered to be correlated with breast cancer progression, one aspect of the present invention is directed to measuring endogenous ERK7/8 levels as a diagnostic and therapeutic indicator of cancer and cancer progression. In one embodiment a method of diagnosing the presence of an estrogen responsive cancer in a patient is provided, wherein the method comprises the step of determining ERK7/8 levels in the cells of a biological sample obtained from said patient; and determining whether the ERK7/8 levels are below a threshold level. The expression levels of ERK8 protein in various breast cancer cells has been quantitated and normalized to the ERK8 protein levels observed in normal breast tissue (see FIG. 4). As shown in FIG. 4, relative to non-cancerous and benign tumors seven different cancers have significant reduced ERK8 protein levels.

In accordance with one embodiment a method of early detection of estrogen receptor associated cancers, and more particularly breast cancer, in patients is provided. The method comprises the step of determining ERK8 levels in the cells of a biological sample obtained from said patient, comparing the ERK8 expression in said sample cells to ERK8 expression in non-cancer cells, wherein a decrease of greater than 20% relative to the non-cancer cell levels is a diagnostic indication that the patient has cancer. In one embodiment the ERK8 level to be measured is the relative ERK8 protein concentration, in an alternative embodiment the ERK8 level to be measured is the relative ERK8 kinase activity. The ERK8 levels in the patient's biological sample are compared to a reference “normal” level established for non-cancerous cell. Preferably, the reference sample is based on non-neoplastic cells of the same tissue type as the biological sample (i.e. biopsy) recovered from the patient. In one embodiment the reference normal ERK8 levels are established based on a large population of cells recovered from multiple healthy individuals. In one embodiment a reduction of greater than 50% in ERK8 levels is indicative of the presence of cancer cells in said patient, in another embodiment a reduction of 30%, 40%, 50%, 60%, 70% or greater is indicative of cancer.

Typically the biological sample used to measure ERK8 levels is recovered from the patient in the form of a tissue biopsy. However, the biological sample may be recovered by less intrusive means including drawing blood from the patient and measuring the ERK8 levels of blood cellular components.

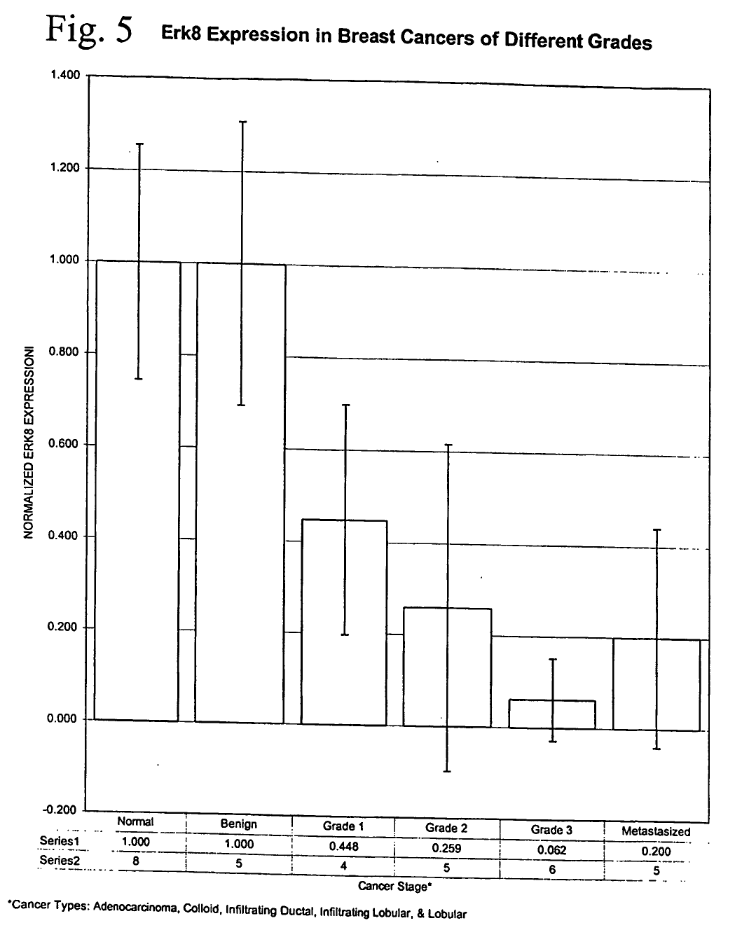

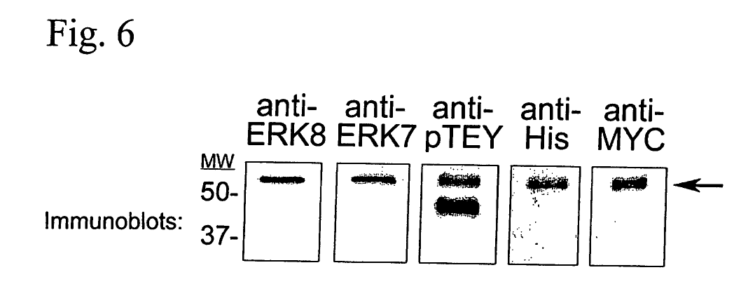

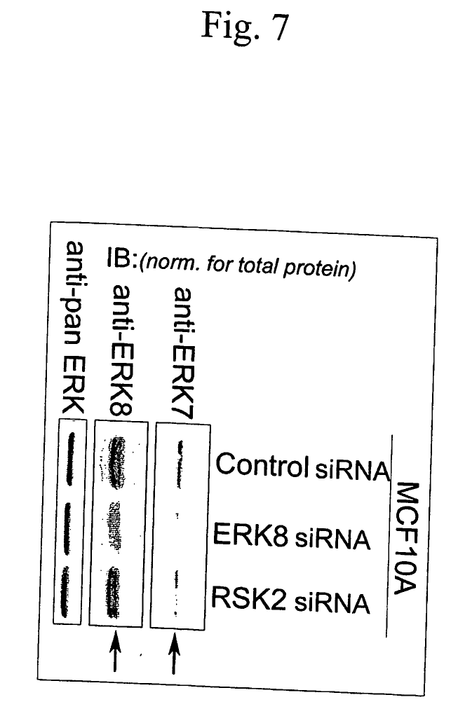

As indicated in FIG. 5, ERK8 levels continue to decrease in more severe grades of cancer. According, in one embodiment of the present invention ERK8 levels are used to characterize the aggressiveness or severity of the tumor to help determine treatment therapy strategies. The method for diagnosing and determining the prognosis of a cancer patient comprises the step of determining the level of ERK8 expression in a biopsy sample, wherein the severity of the decrease in ERK8 expression is correlated with the stage of the cancer. Furthermore, to assist in determining the prognostic outlook and help define therapeutic strategies, estrogen receptor alpha (ER α) levels in the biological sample can also be measured. Increased estrogen receptor alpha (ER α) levels (relative to healthy population levels) in combination with below normal ERK8 levels indicate a more advanced stage of cancer.

In a further embodiment ERK8 levels (i.e. either nucleic acid or protein concentration or ERK8 kinase activity) or the rate of endogenous ERα degradation can be monitored as an indicator of the effectiveness of an anticancer therapy. Successful therapy would be indicated by either an increase in ERK8 levels or by an increase in endogenous ERα degradation during therapy. Monitoring ERK8 levels in estrogen responsive cancer cells cultured in vitro can also be used to identify the effectiveness of anti-cancer agents and help identify new anti-cancer agents. In accordance with one embodiment a method of monitoring the effectiveness of an anti-cancer agent for treating estrogen responsive cancers is provided. The method comprises the steps of monitoring ERK8 levels in estrogen responsive cancer cells contacted with one or more anti-cancer agents. The cancer cells may come in contact with the anti-cancer agent in vivo during the course of treating a cancer patient or alternatively the cancer cells may be culture in vitro and contacted with the anti-cancer agent. In accordance with one embodiment the cancer cells to be contacted in vitro with the anti-cancer agent are selected from various established tumor cell lines. In another embodiment the cancer cells to be contacted in vitro with the anti-cancer agent are recovered from a patient and treated in vitro, as a means of optimizing or identifying the best anti-cancer therapy.

In one embodiment, the ERK8 levels are determined using antibodies specific for ERK8, typically in an ELISA assay, immunohistochemical analysis or through use of flow cytometry utilizing antibodies or fluorescent, covalently-bound small molecules which specifically interact with ERK8. Polyclonal antibodies raised against rat ERK7 have been found to also recognize human ERK8 as shown using recombinant ERK8 and by siRNA performed in MCF-10A cells. In accordance with one embodiment, below average ERK8 protein levels are detected through the use of a capillary fill device. In one embodiment the capillary fill device comprises a base provided with a capillary space, said capillary space having an interior surface and a first and second end;

a first port formed on an exterior surface of said base and in fluid communication with the first end of said capillary space;

a second port formed on an exterior surface of said base and in fluid communication with the second end of capillary space;

a reaction zone and a detection zone each formed on said interior surface of the capillary space, wherein said reaction zone comprises a labeled ERK8 antibody and the detection zone is located more proximal to the second port than the reaction zone, wherein fluid introduced into said first port will travel through the capillary space, contacting the reaction zone and then the detection zone as the fluid moves to the second port. Preferably the ERK8 is deposited on the reaction zone in a manner that contact with fluid moving through the capillary space releases the antibody and allows the antibody to specifically bind to any corresponding target antigen present in fluid. Accordingly when a fluid derived from a biological sample is placed in the first port the fluid enters the capillary space and resuspends the ERK8 antibody which then binds to any ERK8 present in the biological sample fluid. The fluid then comes in contact with the detection zone wherein the amount of ERK8 present in the biological sample derived fluid is determined. In one embodiment the detection zone comprises a second antibody specific for ERK8 (preferably binding an ERK8 epitope separate and distinct from the epitope that the first ERK8 antibody binds), wherein the second antibody is linked to capillary inner surface and immobilizes any ERK8 antibody/ERK8 complexes coming in contact with the second antibody. In this embodiment the signal generated by the entrapped labeled ERK8 antibody/ERK8 complex corresponds to the concentration of ERK8 within the fluid administered to the device.

In one embodiment, the present invention is directed to antibodies that specifically bind to ERK7 or ERK8. These antibodies are used to detect ERK8 present in human derived biological tissues. In one embodiment, ERK8 antibodies are generated that bind to full length ERK8 but not a truncated mutant of ERK8, such as a polyclonal antibody to the extreme C-terminus of ERK8. In another embodiment, ERK8 antibodies may also detect both active and inactive ERK8, such as anti-AL ERK8, which recognizes residues in the activation loop of ERK8. In one embodiment the antibodies are generated to the sequences CRSALGRLPLLPGPRA (SEQ ID NO: 4) and CQALTEY (SEQ ID NO: 5), and in a further embodiment the antibodies are monoclonal antibodies. These antibodies may be used as diagnostic or therapeutic markers for cancer.

Antibodies to ERK7 and ERK8 polypeptides or peptide fragments thereof may be generated using methods that are well known in the art. For the production of antibodies, various host animals, including but not limited to rabbits, mice, rats, etc can be immunized by injection with an ERK7/8 polypeptide or peptide fragment thereof. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of ERK8 polypeptides together with genes from a human antibody molecule of appropriate biological activity can be used; such antibodies are within the scope of this invention.

According to one embodiment of the invention, techniques described for the production of single chain antibodies (U.S. Pat. No. 4,946,778) can be adapted to produce protein-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for sperm surface proteins, derivatives, or analogs.

Antibody fragments which contain the idiotype of the molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay). Antibodies generated in accordance with the present invention may include, but are not limited to, polyclonal, monoclonal, chimeric (i.e. “humanized” antibodies), single chain (recombinant), Fab fragments, and fragments produced by a Fab expression library.

The antibodies of the present invention can be combined with a carrier or diluent to form a composition. In one embodiment, the carrier is a pharmaceutically acceptable carrier. In another embodiment the antibody is linked to a solid support, and in a further embodiment the antibody is releasably bound to a solid support, using standard techniques known to those skilled in the art. For example, the antibody can be simply adhered to the surface of the support by drying a liquid composition comprising the antibody, or alternatively the antibody can be covalently bound through either an enzymatically cleavable or photoliable linker moiety or the polypeptide can be bound through ionic interactions and subsequently released by changes in salt or pH conditions. In another embodiment the antibody is linked to a detectable marker.

The ERK7 and ERK8 antibodies of the present invention can be labeled using standard techniques and detectable markers known to those skilled in the art. The label can be either directly linked or indirectly linked to the ERK7/8 antibody. In the indirect method, the detectable marker is attached to a secondary antibody that recognizes the ERK7/8 specific monoclonal antibody. The indirect method has the advantage that it can amplify the signal by binding more of the detectable marker at the antigen site, thus its potential signal on its target may be stronger than the direct method, especially at low antibody-conjugate concentrations. A drawback of this method is that it employs two separate steps of antibody addition. The direct method has the advantage that it reduces the number of washing steps and is quicker. The use of a single labeled immunoreagent also reduces the background by eliminating non-specific binding of the secondary antibody.

In accordance with one embodiment the ERK7/8 antibody is labeled and used in a device to measure the amount of ERK7/8 present in a biological sample. In one embodiment the ERK7/8 antibody is a monoclonal antibody. Such antibodies have utility as diagnostic markers for cancer as well as utility in therapeutic applications.

In one embodiment, the present invention provides methods of screening for agents, small molecules, or proteins that interact with polypeptides comprising the sequence of SEQ ID NO:1 or a fragment thereof. The invention encompasses both in vivo and in vitro assays to screen small molecules, compounds, recombinant proteins, peptides, nucleic acids, antibodies etc. which bind to or modulate the kinase activity of ERK7 and ERK8. Modulators of ERK7 and ERK8 are anticipated to have therapeutic use.

In one embodiment of the present invention ERK8, or fragments thereof are used to isolate ligands that bind to ERK8 under physiological conditions. The method of isolating such ligands comprises the steps of contacting an ERK8 polypeptide with a mixture of compounds under physiological conditions, removing unbound and non-specifically bound material, and isolating the compounds that remain bound to the ERK8 polypeptide. Typically, the ERK8 polypeptide will be bound to a solid support, using standard techniques, to allow for rapid screening of compounds. In one embodiment the ERK8 polypeptide is releasably bound to a solid support, using standard techniques known to those skilled in the art. For example, the polypeptide can be covalently bound through either an enzymatically cleavable or photoliable linker moiety or the polypeptide can be bound through ionic interactions and subsequently released by changes in salt or pH conditions. The solid support can be selected from any surface that has been used to immobilize biological compounds and includes but is not limited to polystyrene, agarose, silica or nitrocellulose. In one embodiment the solid surface comprises functionalized silica or agarose beads. Screening for such compounds can be accomplished using libraries of pharmaceutical agents and standard techniques known to the skilled practitioner.

Ligands that bind to the ERK7 and/or ERK8 polypeptides can then be further analyzed for agonists and antagonists activity through the use of an in vitro kinase assay such as that described in Example 2. Compounds that enhance the activity of ERK8 have potential use as agents for treating cancers that are responsive to estrogen. These include, but are not limited to, breast, ovarian, prostate, endometrial, colon, pancreas and brain cancers and neoplastic tumors. In one embodiment compounds are screened for their ability to modulate ERK7 and/or ERK8 kinase activity, and in one embodiment compounds are screened for their ability to enhance ERK8's kinase activity.

In accordance with one embodiment, a method for identifying modulators of ERK7/8 activity is provided. The method comprises the steps of

contacting an ERK7/8 polypeptide with a kinase substrate, and ATP, to form a control mixture;

contacting an ERK7/8 polypeptide with a kinase substrate, ATP, and a potential ERK7/8 activity modulator, to form a reaction mixture;

incubating the control and reaction mixtures under identical conditions for a predetermined length of time; and

comparing the amount of phosphorylated substrate produced in the reaction mixture to that produced in the control mixture, wherein a different concentrations of phosphorylated substrate produced by the control and reaction mixtures identifies a ERK7/8 activity modulator. In accordance with one embodiment compounds are screened for their ability to enhance ERK7/8's ability to phosphorylate its substrate relative to an ERK7/8 kinase reaction run in the absence of the compound. In another embodiment compounds are screened for their ability to inhibit ERK7/8's ability to phosphorylate its substrate relative to an ERK7/8 kinase reaction run in the absence of the compound. Compounds that enhance ERK8's kinase activity can be formulated as pharmaceutical compositions and administered to a subject to treat cancer and neoplastic tumors for estrogen responsive neoplastic growths. In advanced stage and aggressive cancers ERα may be at such low levels that treatment with an ERK8 kinase inhibitor may be advantageous to maintain the cancer in a state that is responsive to anti-hormonal therapy.

### Example 1

Erk7 Specifically Enhances ERα Destruction

Materials and Methods

Reagents

The monoclonals, 12CA5 (anti-HA) and 9e10 (anti-MYC), were obtained from the Univ. Virginia Lymphocyte Culture Facility. The anti-active MAPK antibody (also called anti-pTEY antibody) was purchased from Promega. The cDNAs for human ERα, human IκBα, human AR, and mouse SF1 were provided by P. Chambon (EMBO J. 8:1981-1986.), D. Ballard (Mol Cell Biol. 15:2809-2818), M. Weber (J Biol Chem. 277:29304-29314) and K. Parker (Mol Endocrinol. 9:1233-1239), respectively. MEKc was provided by N. Ahn (J Biol. Chem. 267:25628-25631), ERK5 by J. Dixon (J Biol Chem. 270:12665-12669) and HAubiquitin by D. Bohmann (Cell. 78:787-798). The peptides to the C-terminal end of rat ERK7 (CRSALGRLPLLPGPRA; SEQ ID NO: 4) and to the activation loop of rat ERK7 (CQALTEY; SEQ ID NO: 5) and the polyclonals, anti-ERK7 and anti-AL ERK7, were produced by Research Genetics.

Expression Vectors

The cDNA for ERK7 was obtained from a rat testis cDNA library using the polymerase chain reaction (PCR) with primers specific to the published ERK7 sequence (Mol Cell Biol. 19:1301-1312). The sequence was verified and the cDNA was inserted in frame into pK3H (contains a triple HA tag) or PKR7-MYC (contains a MYC tag) or modified pCMV-Tag5 (Stratagene). The cDNA for ERK8 was obtained from a human EST library. The sequence was verified and the cDNA was inserted in frame into pK3H or PKR7-MYC or modified pCMV-Tag5. The vector pCMV-Tag5 (Stratagene) was modified by inserting the promoter and triple HA tag sequences from pKH3 into pCMV-Tag5. All ERα and ERK7 and ERK8 mutants were produced using PCR and the sequences verified by the Univ. Virginia Biomolecular Research Facility. Both w.t. and mutant ERαs were sub-cloned into pSG5.

Immunoblots

BHK cells were transfected in 10 mm dishes with calcium phosphate and 1 μg of wild type or mutant ERα or FLAG-IκBα or HA-SF1 or HA-AR construct, 5 μg of a construct encoding a wild type or mutant kinase or vector control. At 8 h post transfection, the cells were washed with phosphate-buffered saline (PBS) and placed in Dulbecco's modified Eagle's medium (DMEM) with 5% fetal calf serum (FCS). After 1 h the cells were washed with PBS, serum starved for 15 h and either estradiol or vehicle added 6 h before lysis with boiling SDSPAGE sample buffer (−DTT, see Joel et al., (1998) J Biol Chem. 273:13317-13323). For the rate experiments transfected cells were trypsinized, aliquoted and pre-treated with 50 μM cycloheximide for two hours in the absence of serum before the addition of estradiol. The cells were lysed at various times after estradiol treatment.

Breast lines were serum-starved for 15 h before lysis with sample buffer (−DTT). Human tissue obtained from the University of Virginia Tissue Procurement Facility was pulverized in the presence of liquid N₂ and the powder added to sample buffer (−DTT). Total protein was determined using DC protein assay (Bio-Rad) and 100 mg of protein electrophoresed (DTT was added to each sample prior to loading) and transferred to nitrocellulose. Immunoblots and densitometric analysis were as described (J Biol Chem. 273:13317-13323).

Immunoprecipitations

BHK cells were transfected in 150 mm dishes with calcium phosphate and 2.5 μg of a ubiquitin construct and either 22.5 μg of MYC-ERK7 or vector control. Additionally, the cells were co-transfected with 2.5 μg of MYC-ERα(282-595) or additional vector control. The transfected cells were washed and then serum starved. Lysis and immunoprecipitation with 12CA5 were as described (EMBO J. 20:3484-494).

Generation of Stable Clones

MCF-7 cells were maintained in DMEM with 5% charcoal-stripped (cs) FCS and 5% CO₂. They were transfected with Lipofectamine (Life Technologies) according to the manufacturer's directions with 6 μg of the modified pCMV-Tag5 vector with or without the K43A-ERK7 cDNA or ERK8 cDNA. Stable clones were obtained using G418 (600 mg/ml) selection. Isolated colonies were cloned, propagated and where appropriate the lines were analyzed for expression of HA-K43A-ERK7.

Transcriptional Analysis

MCF-7 stables were transfected and assayed for luciferase and β-galactosidase as described previously (EMBO J. 20:3484-494).

Proliferation

MCF-7 stables were seeded at 5×10⁴ in a 24 well dish containing DMEM and 5% csFCS with 600 μg/ml G418. After 15 hrs the cells were treated with or without 1 μM ICI 182,780. At various times the cells were lysed and the amount of ATP determined by CellTiter-Glo™ Luminescent Cell Viability Assay according to the manufacturer's protocol (Promega).

Results

Erk7 Specifically Enhances ERα Destruction

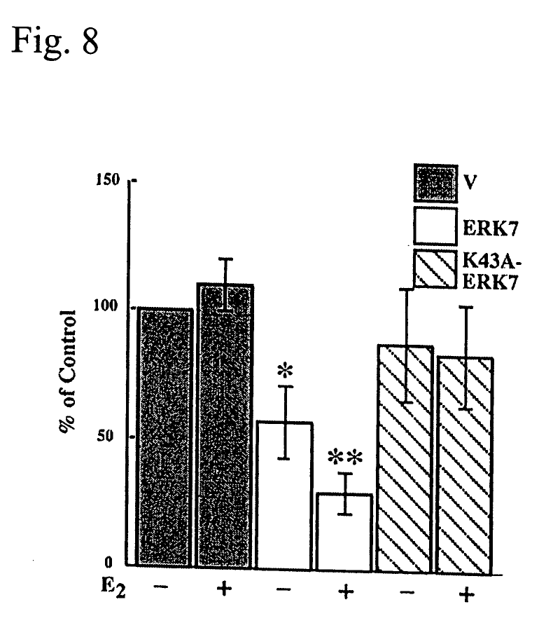

To test whether members of the MAPK(ERK1/2) family regulate ERα turnover, family members were investigated for their ability to influence the steady state levels of ERα expression. ERα and the indicated kinases were co-expressed in baby hamster kidney (BHK) cells and the expression levels of ERα and the kinases determined by immunoblotting the lystates of the transfected cells. In these experiments BHK cells were chosen because they are easily transfected and support ERα-mediated transcription when provided with ERα cDNA. Ectopic expression of MAPK(ERK1/2) would not substantially increase cellular MAPK(ERK1/2) activity because in order for MAPK(ERK1/2) to be active it must be phosphorylated by MAPK kinase (MEK). Therefore, a constitutively active mutant of MEK (MEKc) was expressed to activate endogenous MAPK(ERK1/2). In this transient transfection system we did not observe any difference in the steady state levels of ERα between vehicle and estradiol treatments in the vector control (FIG. 8). However, in the vector control, the amount of ERα present in the slower electrophoretic migrating band increased in the presence of estradiol. We previously determined that phosphorylation of Ser-118 in human ERα results in a reduced electrophoretic mobility. Activation of endogenous MAPK(ERK1/2) by MEKc increased the amount of ERα present in the slower migrating band, which can be most clearly observed by comparison with the vector control in the absence of estradiol. These data are consistent with observations that MAPK(ERK1/2) phosphorylates Ser-118.

The antipTEY antibody (also referred to as the anti-active MAPK antibody) specifically recognizes the dual-phosphorylated Thr and Tyr residues present in the activation loop of MAPK(ERK1/2) and ERK7. Thus, as expected, the anti-pTEY immunoblot shows that the expression of HA-MEKc enhanced the activity of MAPK(ERK1/2) above that observed with the vector control. However, increasing MAPK(ERK1/2) activity did not alter ERα protein levels compared to the vector control. Remarkably, however, the ectopic expression of ERK7 decreased the steady state levels of ERα expression ˜2-fold in the absence and ˜3-fold in the presence of estradiol (FIG. 8). The immunoblot with the anti-pTEY antibody demonstrates that similar levels of active ERK7 and endogenous MAPK(ERK1/2) were achieved. Expression of another member of the TEY kinase subgroup, ERK5, did not influence ERα levels, further demonstrating that a decrease in the steady state level of ERα is a specific response to ERK7.

Steady state levels reflect both the rates of synthesis and degradation. Therefore, to determine the effect of ERK7 on the rate of ERα destruction we blocked protein synthesis using the inhibitor, cycloheximide, and determined the amount of ERα by densitometry at various times after the addition of vehicle or estradiol. To eliminate transfection differences between time points the BHK cells were distributed into the appropriate number of plates after being transfected. The rate data were fitted using a single exponential decay and the rate constants determined. Based on these rate constants, the t1/2 for ERα in BHK cells is ˜4 hrs and in the presence of estradiol the t1/2 is ˜3 hrs. In BHK cells ectopic expression of ERK7 decreases the t1/2 to ˜2.5 hrs and ˜1.9 hrs in the absence and presence of estradiol, respectively. These data also demonstrate that the decrease in ERα levels in the presence of ERK7 is not a transfection artifact because the rate data are independent of transfection efficiency.

To understand the role that the kinase activity of ERK7 plays in enhancing ERα destruction the ability of the mutant K43A-ERK7 to influence ERα steady state levels was determined. In this mutant the essential Lys-43, necessary for ATP hydrolysis, has been changed to Ala. K43A-ERK7 does not have catalytic activity in an in vitro kinase assay using myelin basic protein as a substrate, in agreement with Abe et al. (Mol Cell Biol. 19:1301-1312) who found that mutating Lys-43 to Arg destroyed ERK7 catalytic activity. Co-expression of ERα with kinase-dead ERK7 (HA-K43A-ERK7) did not significantly decrease ERα protein levels compared to the vector control (FIG. 8). Similar effects were also observed with a kinase-inactive ERK7 in which the Thr and Tyr present in the TEY phosphorylation motif were mutated to Ala. Wild type and kinase-dead ERK7 were expressed to similar extents but only wild type ERK7 is able to decrease ERα levels. Therefore, these data demonstrate the kinase activity of ERK7 is required to decrease ERα protein levels.

ERK7 Specifically Enhances ERα Degradation.

ERα is degraded through the 26S proteasome pathway, and one possible explanation for the observed decrease in ERα levels is that ERK7 enhances the rate of proteasome-mediated degradation. Therefore, the ability of the competitive proteasome inhibitor, MG132, to prevent ERK7 from decreasing ERα levels was tested. In agreement with the results of Nawaz et al., addition of MG132 increased ERα protein levels in both the presence and absence of estradiol. Treatment with MG132 also increased the amount of ERα co-expressed with ERK7, and increased the abundance of higher MW forms of ERα induced by ERK7. In the presence of a proteasome inhibitor the ubiquitinated products accumulates and thus the higher MW ERα forms are likely due to ubiquitination. Similar results were observed with another proteasome inhibitor, lactacystin. It is not possible to determine by immunoblotting whether these forms are a result of ubiquitination because anti-ubiquitin antibodies are extremely insensitive. Additionally, our anti-ERα antibody does not immunoprecipitate the higher MW ERα forms. These results suggest that ERK7 decreases the abundance of ERα by enhancing its degradation through the proteasome pathway. To test the specificity of ERK7-mediated degradation we determined whether ERK7 could enhance the destruction of IκBα, which is degraded by the 26S proteasome pathway. ERK7 did not reduce the protein levels of IκBα in comparison to the vector control. These results support the hypothesis that ERK7 is a specific regulator and is not a general activator of the ubiquitination/proteasome machinery.

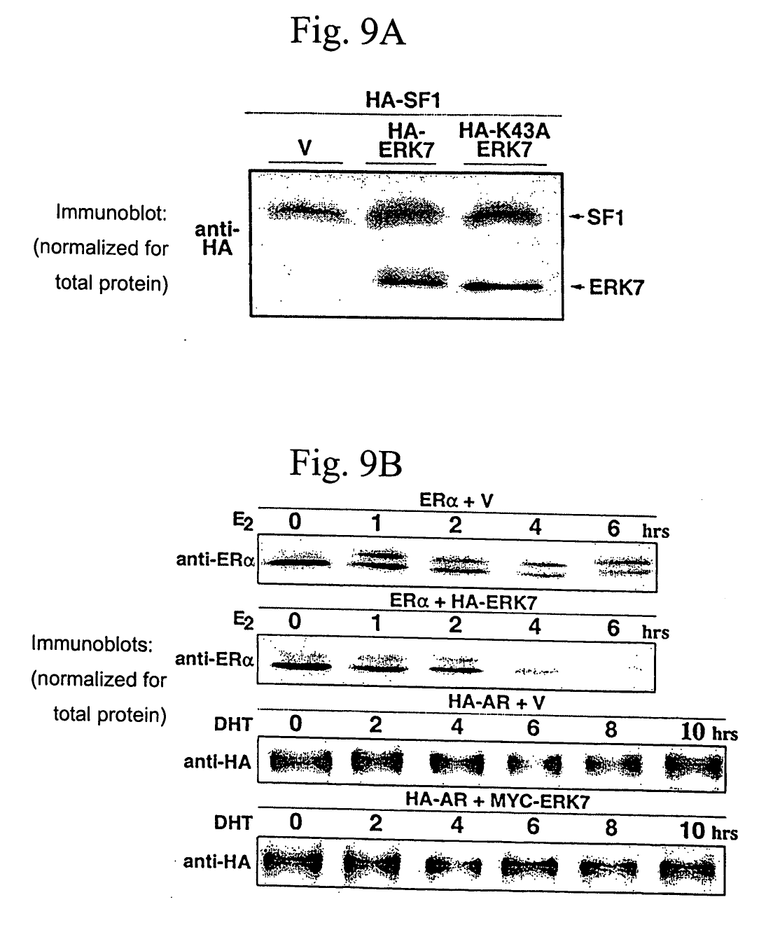

To determine whether ERK7 can trigger the destruction of other nuclear receptor superfamily members, ERK7's effect on steroidogenic factor I (SF1), a distant, evolutionarily-related relative of ERα, was investigated. ERK7 had no effect on the protein levels of SF1 compared to the vector control (FIG. 9A). These results suggest that ERK7 stimulates the degradation of only a subset of the nuclear receptor superfamily, which includes ERα. To further define the nuclear receptors that are targets of ERK7 we tested the ability of ERK7 to enhance the rate of degradation of the androgen receptor (AR) a close relative of ERα. The rate of degradation was determined in the presence of cycloheximide in a similar manner to that performed with ERα, except that dihydryoxytestosterone was used. Remarkably, ERK7 did not enhance the degradation of the AR (FIG. 9B). Taken together, these data suggest that ERK7 specifically regulates ERα turnover.

Erk7 is Highly Expressed in Normal Human Breast Cells.

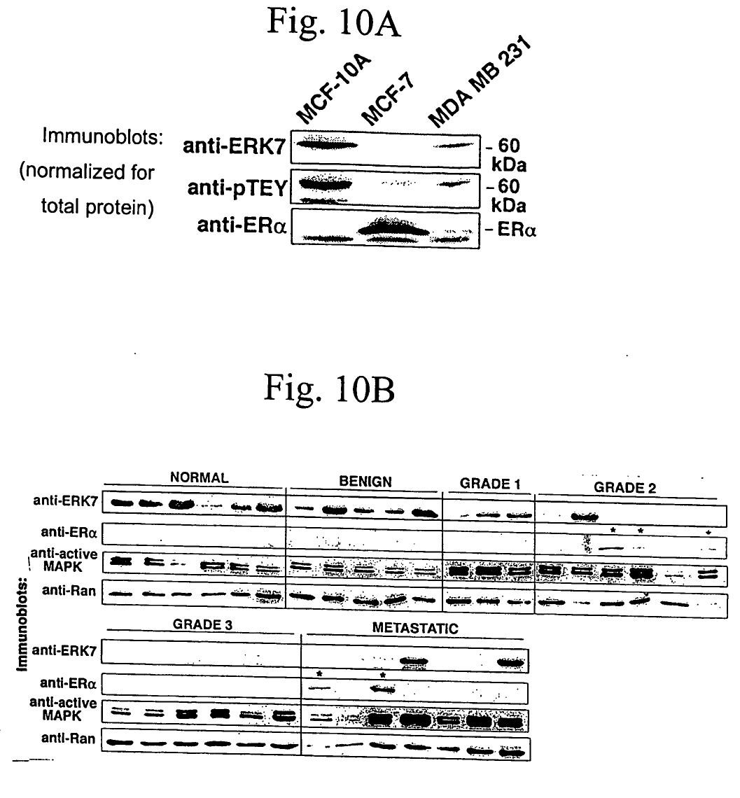

To understand the importance of ERK7 in regulating estrogen action ERK7 expression in breast cells, an estrogen target tissue, was investigated. A polyclonal antibody was generated to the extreme C-terminus of rat ERK7, which was able to specifically detect ERK7 but not a truncated mutant of ERK7 in lysates from transfected BHK cells. Deletion of the C-terminal tail of ERK7 also inhibits its constitutive kinase activity, and the truncation is therefore not detected by the anti-pTEY antibody. The anti-ERK7 antibody was used to immunoblot lysates of MCF-10A, a normal breast cell line and the breast cancer cell lines, MCF-7 and MDA-MB-231 (FIG. 10A). A band of ˜60 kDa was strongly detected by the anti-ERK7 antibody in MCF-10A cells and this molecular weight (MW) is in agreement with the calculated MW based on the rat ERK7 cDNA. The ˜60 kDa band recognized by anti-ERK7 co-localized with a band recognized by the anti-pTEY antibody (FIG. 10A). Together, these data strongly suggest that the ˜60 kDa band observed in normal breast cells is human ERK8. It is not particularly surprising that the antibody raised to rat ERK7 recognizes human ERK8 because a number of kinases are virtually identical in their amino acid sequences between rats and humans, eg. ERK2, and as demonstrated in FIG. 3 rat ERK7 and human ERK8 share a high degree of sequence similarity.

The MCF-10A cell line has higher levels of ERK7 than the MCF-7 and MDA-MB-231 cell lines. The amount of ERα in the different cell lines is inversely correlated with the amount of ERK7. The amount of ERα is extremely low in MCF-10A and can only be detected by immunoblotting anti-ERα immunoprecipitations. These results are consistent with the idea that ERK7 regulates ERα turnover, because the MCF-10A cell line has low levels of ERα and the MCF-7 cell line has high levels of ERα. The MDA-MB-321 cell line represents an intermediate between MCF-10A and MCF-7. To further understand the physiological significance of ERK7 in regulating estrogen action the expression levels of ERK7 in normal human breast tissue and breast cancer tissue was determined (see FIG. 10B and FIG. 4). Detection of the 60 kDa band in normal human breast tissue was blocked by pre-incubating the anti-ERK7 antibody with the antigenic peptide. In total 13 normal tissues, 5 benign tumors, and 66 breast tumors were examined and the results shown are representative. In FIG. 5 the data is presented by grouping the samples according to their tumor grade from the least to the most aggressive according to the pathologist's report. The tissue samples were normalized to each other by immunoblotting for Ran, a housekeeping protein whose expression level is not known to change with any disease state.

ERK7 was expressed in all of the normal and benign tissue samples, ˜half of the grade 1 samples, ˜20% of the grade 2 tumors, none of the grade 3 tumors and ˜30% of the metastatic tumors (FIG. 10B). Thus loss of ERK7 is correlated with breast cancer progression. These samples were also analyzed for active MAPK(ERK1/2) and in agreement with the literature MAPK(ERK1/2) activity was generally higher in breast cancers than in the normal or benign tumor samples. But, importantly, a correlation between MAPK(ERK1/2) activity and loss of ERK7 was not observed. These results demonstrate that the loss of ERK7 is not merely a reflection of a general decrease in the levels of various members of the MAPK(ERK1/2) family. Also in agreement with the literature normal human breast mammary epithelial cells were observed to have very low levels of ERα, which is consistent with the data presented herein that normal breast tissue has significant amounts of ERK7. Additionally, cancer tissues that had detectable amounts of ERα had undetectable levels of ERK7 expression. Thus loss of ERK7 correlates with increased ERα levels. These data further suggest that ERK7 is an important regulator of estrogen action in the breast.

ERK7 Regulates Hormone Responsiveness in Human Breast Cells

The concentration of ERα is limiting for estrogen responsiveness in vivo, and ERα synthesis and degradation, therefore, play a pivotal role in controlling the expression of ERα-regulated genes. Taken together, the present results indicate that ERK7 regulates hormone responsiveness in cells that endogenously express ERα. To test this hypothesis, the creation of MCF-7 clones that stably expressed HA-ERK7 was attempted, however, the majority of the synthesized ERK7 was not active. These results suggest that ERK7 activity is regulated differently in breast cells than in BHK cells in which ˜50% of ERK7 is active. Kinase-dead mutants are often able to act as a dominant negatives and therefore, MCF-7 clones were produced that stably expressed K43A-ERK7. The hypothesis was that the MCF-7 cells contained extremely low levels of ERK7 and that the cell's ability to degrade ERα be inhibited by using K43A-ERK7. In MCF-7 lysates a faint band at ˜60 kDa was observed upon extended exposure of the anti-ERK7 immunoblot. The anti-ERK7 antibody is able to immunoprecipitate HA-ERK7 from lysates of transfected cells and therefore, this antibody was used to immunoprecipitate ERK7 from MCF-7 lysates. ERK7 was detected in the inimunoprecipitate using another polyclonal antibody to ERK7 that recognizes residues in the activation loop of ERK7 (anti-AL ERK7). The anti-AL ERK7 recognizes both the active and inactive forms of ERK7. Thus these results demonstrate that MCF-7 cells do contain ERK7. In the K43A-ERK7 stable cell lines, addition of cycloheximide decreased the degradation of ERα by ˜1.5 fold and >4 fold in the absence and presence of estradiol, respectively, compared to that in the vector stable lines. Thus K43A-ERK7 is more effective at inhibiting the degradation of the hormone-bound receptor than that of the unbound receptor. Over the 4 hr cycloheximide treatment, the level of K43A-ERK7 decreased significantly, which suggests that ERK7 is turned over rapidly in MCF-7 cells. It is likely that the effects on ERα degradation would have been much more dramatic had the levels of K43AERK7 remained constant over the time course of the experiment. These results suggest that K43A-ERK7 is able to act as a dominant negative in MCF-7 cells by inhibiting the action of endogenous ERK7.

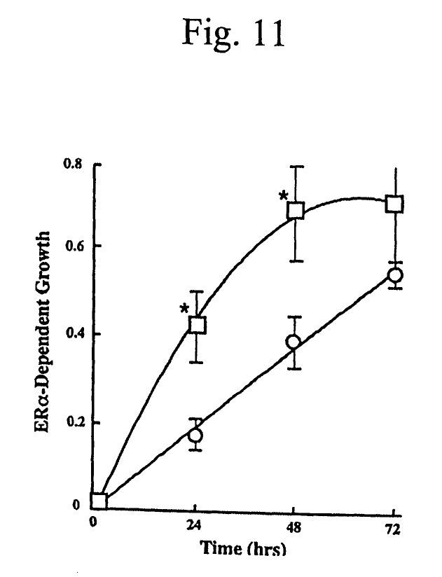

To further test whether ERK7 is an important regulator of estrogen action the effect of K43A-ERK7 on ERα-regulated transcription was investigated. The MCF7 stable lines were cotransfected with a vector encoding the luciferase reporter under the control of estrogen responsive elements (ERE), plus a control vector encoding β-galactosidase. Cells expressing K43A-ERK7 had ˜3 fold greater transcriptional response to estradiol than the control cells. MG132 also increased the transcriptional response of the control cells by ˜3 fold. However, the transcriptional response in the K43A-ERK7 cells was not affected by MG132. These results suggest that K43A-ERK7 and MG132 influence ERα-mediated transcription through a similar mechanism, by inhibiting the ERα degradation rate. In these experiments, a TATA box and a dual ERE regulates luciferase reporter expression. Therefore, ERK7 was tested to determine whether ERK7 also regulates ERα-regulated transcription of endogenous genes in MCF-7 cells. As a readout for broad responses to ERα the ability of estrogen to stimulate MCF-7 cell proliferation was used because the proliferation response is known to be regulated by ERα transcriptional activation. It is likely that ERK7 regulates the activity of molecules in addition to ERα and these other molecules may influence proliferation. Therefore, to examine only the effects of K43A-ERK7 on ERα-mediated proliferation growth was determined in the presence of vehicle or ICI 182,780, a pure estrogen antagonist. To examine only the ERα-dependent growth the growth in the presence of ICI 182,780 was subtracted from that obtained with the vehicle. K43A-ERK7 enhanced the rate of ERα-dependent proliferation ˜2-fold (FIG. 11). These data suggest that ERK7 regulates ERα-mediated transcription from both simple and complex promoters. Furthermore, the ability of K43A-ERK7 to promote proliferation suggests that ERK7 regulation of ERα degradation rate is important in determining estrogen responsiveness.

ERK7 Phosphorylation of ERα does not Influence ERα Stability

Because turnover is commonly regulated by phosphorylation of the target protein, it seemed possible that ERK7 regulates ERα degradation by direct phosphorylation. ERK7 is most likely a proline-directed serine/threonine kinase because the catalytic domain is highly related to MAPK(ERK1/2) and thus there are four putative ERK7 phosphorylation sites in ERα, Ser-104, Ser-106, Ser-118 and Ser-294. It has been previously determined that Ser-118 is the major site of phosphorylation in response to estradiol binding and therefore, whether ERK7 could phosphorylate ERα in vivo was investigated. Lysates from cells transfected with wild type ERα or Ser-118A-ERα in the presence or absence of ERK7 were normalized for total ERα and immunoblotted with anti-ERα. The lysates were normalized to ERα rather than total protein so that the intensities of the various ERα bands between the ERK7 and vector control samples could be directly compared.

The appearance of Ser-118 phosphorylation can be observed as a sharp band and ERK7 enhances the amount of this phosphorylation in both the absence and presence of estradiol. The kinase dead mutant, K43A-ERK7, diminishes the intensity of this slower migrating ERα band. Taken together, these data suggest that ERK7 can regulate-either directly or indirectly—the level of Ser-118 phosphorylation. ERK7 also increases the number of other higher MW ERα forms that are observed as diffuse bands both in wild type ERα and the S118A-ERα mutant.

To determine the role that ERα phosphorylation may play in ERK7-mediated ERα destruction the ability of ERK7 to enhance the rate of S118A-ERα degradation in the presence of cycloheximide was determined. ERK7 was able to enhance the degradation of S118A-ERα with a t1/2 similar to that observed with the wild type ERα. ERK7 was also tested to determine whether ERK7 could enhance the degradation of mutant ERαs that contained mutations in the other putative ERK7 phosphorylation, Ser-104, Ser-106 and Ser-294. There were no significant differences in the ability of ERK7 to decrease wild type ERα protein levels compared to any of the mutants. ERK7 also did not enhance the degradation of a deletion mutant of ERα lacking the ligand-binding domain (ERα (1-282). These data are consistent with those obtained with MEKc in which enhanced Ser-118 phosphorylation by MAPK(ERK1/2) did not influence ERα levels. Thus although ERK7 may enhance Ser-118 phosphorylation it seems that mechanisms other than ERα phosphorylation are important in targeting ERα for destruction.

The ligand-Binding Domain Targets ERα for ERK7-Mediated Destruction

ERK7 targets the wild type ERα for degradation but not a deletion mutant lacking the ligand-binding domain. Therefore, ERK7 was investigated to determine whether it enhanced the destruction of the ligand-binding domain. In agreement with the literature, estradiol was observed to enhanced the level of ERα (283-595) co-expressed with the vector control. Nonetheless, ERK7 was able to decrease the expression level of ERα (283-595) in the presence and absence of estradiol. These results suggest that the ligand-binding domain plays an important role in the ability of ERK7 to regulate ERα protein levels. Inhibitors of the 26S proteasome pathway are able to decrease ERK7-mediated ERα degradation and ERK7 enhances the formation of higher ERα MW forms. Therefore, it is anticipated that the high MW forms consist of ubiquitinated ERα.

However, it is not possible to directly determine whether these higher MW forms are the result of ubiquitination. Therefore, to test the hypothesis that ERK7 enhances ERα ubiquitation, BHK cells were co-transfected with constructs encoding HA-ubiquitin plus either MYC-tagged ERα(283-595) or vector control. Additionally, the cells were transfected with either MYC-tagged ERK7 (MYC-ERK7) or vector control and treated with MG132. To isolate ubiquitinated proteins, lysates were immunoprecipitated with anti-HA antibody, then immunoblotted with either anti-MYC or Ab10, an antibody to the ligand-binding domain of ERα. The amount of ERα(283-595) containing HA-ubiquitin was greatly increased in the presence of ERK7 compared to the vector control. Bands were identified at a MW consistent with the addition of a single ubiquitin to MYC-ERα(283-595), whereas the other bands were observed consistent with polyubiquitination. The results with Ab10 were similar to those obtained with the α-MYC antibody). These results support the hypothesis that ERK7 enhances the level of ERα ubiquitination.

Discussion

It has been previously reported that, in response to hormone, the phosphorylation of Ser-118 increases. ERK7 enhances Ser-118 phosphorylation in vivo but this phosphorylation is not important for ERK7-mediated degradation because ERK7 is able to enhance the rate of degradation of ERα and S118-ERα to the same extent. In addition, no evidence was uncovered that mutation of other putative ERK7 phosphorylation sites influenced ERK7-mediated ERα degradation. It seems unlikely that phosphorylation of the ligand-binding domain plays a role in regulating turnover because enhanced phosphorylation in the ligand-binding domain has not been observed using (³²P)-orthophosphate labeling and microsequencing of the radiolabeled ERα peptides in the absence or presence of lactacystin.

There are several cases known in which the destruction of specific targets is regulated by kinases. The kinases phosphorylate a residue within a specific sequence context, and this phosphorylation results in the interaction with particular E2 and E3 enzymes. For example, glycogen synthase kinase −3β phosphorylates β-catenin and IκB kinase phosphorylates IκBα, which regulates their destruction through the Skp1-Cullin-F-box complex. There is also evidence that the destruction of some members of the nuclear steroid receptor superfamily is regulated by their phosphorylation. However, the discovery of ERK7-mediated destruction of ERα is mechanistically distinct. One possible mechanism is that ERK7 phosphorylates a component of the ubiquitin machinery, which increases its catalytic activity or affinity for ERα. Another plausible mechanism is that ERK7 phosphorylates an ERα-interacting protein, which exposes a surface on ERα that targets it for destruction.

Remarkably, ERK7 expression is lost during breast cancer progression. Normal breast epithelia cells have extremely low levels of ERα and are considered to be ERα-negative. Hormone-responsive breast cancers express 10-fold or more higher ERα levels but as the cancer becomes more aggressive ERα expression is frequently lost. In our studies all the ERα positive tumors also had lost ERK7 expression and it may be that the increase in ERα levels in hormone responsive breast cancers is due to decreased ERK7 expression. However, not all tumors that had lost ERK7 expression were ERα positive, suggesting that other factors in addition to ERK7 are involved in regulating ERα levels. It may be that during tumor progression ERK7 downstream effectors have been inappropriately activated, resulting in reduced ERα levels even in the absence of ERK7. Accordingly it is believed that ERK7 is important in maintaining the homeostasis of a normal breast cell and that with high frequency ERK7 is lost at an early step in breast cancer progression.

### Example 2

Assay for Measuring ERK7 & 8 Activity

Kinase assays were developed to measure the activity of rat ERK7 and human ERK8. Purified, recombinant ERK7 and ERK8 were obtained by introducing a HIS-tag in frame with the coding sequence of the kinases, expressing the proteins in bacteria and purifying the proteins over a NiNTA resin (Qiagen, Valencia, Calif.). Immunoprecipitated ERK7 and ERK8 were obtained by introducing a HA-tag in frame with the coding sequence of the kinases, expressing the proteins in baby hamster kidney cells and immunoprecipitating and eluting the kinases off the beads using an HA-peptide.

The kinase assays were performed in a 96 well format according to the following procedure. Glutathione-S-transferase (GST)-fusion protein (1 μg/well) containing the sequence—KNIVTPRTPPPSQGKG (SEQ ID NO: 6; corresponding to human myelin basic protein) (GST-MBPtide) was adsorbed in the wells of LumiNunc 96-well polystyrene plates (MaxiSorp surface treatment). The wells were blocked with sterile 3% tryptone in phosphate buffered saline and stored at 4° C. for up to 6 months. ERK7 or ERK8 (5 nM of purified, bacterially expressed or kinase immunoprecipitated from approximately 10% of a confluent 15 cm tissue culture plate) in 70 μl of kinase buffer (5 mM β-glycerophosphate pH 7.4, 25 mM HEPES pH 7.4, 1.5 mM DTT, 30 mM MgCl₂, 0.15 M NaCl) was dispensed into each well. Reactions were initiated by the addition of ATP and terminated after 30 to 60 min by addition of 75 μl 500 mM EDTA, pH 7.5. All assays measured the initial velocity of reaction. After extensive washing of wells, monoclonal phosphospecific antibody developed against the phosphopeptide (anti-Phospho MBP, Upstate Biotechnology, catalog # 05-429) and HRP-conjugated anti-mouse antibody (115-035-146, Jackson ImmunoResearch Laboratories, West Grove, Pa.) were used to detect threonine phosphorylation of the substrate. HRP activity was measured using Western Lightning Chemiluminescence Reagent (NEL102, PerkinElmer Life Sciences) according to manufacturer's protocol. Maximum and minimum activity is the relative luminescence detected in the presence of vehicle and 200 mM EDTA, respectively.

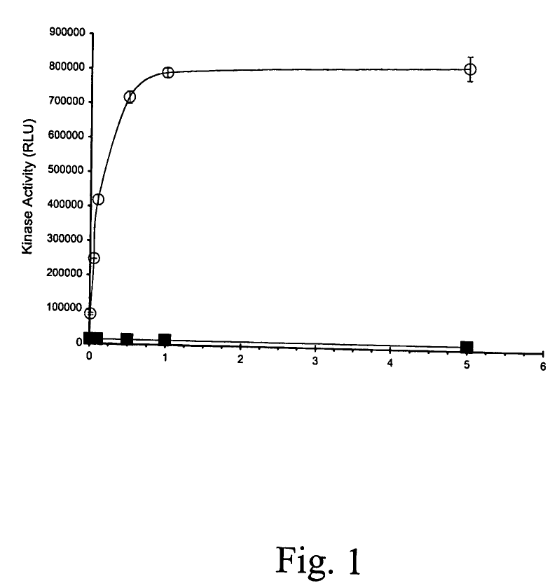

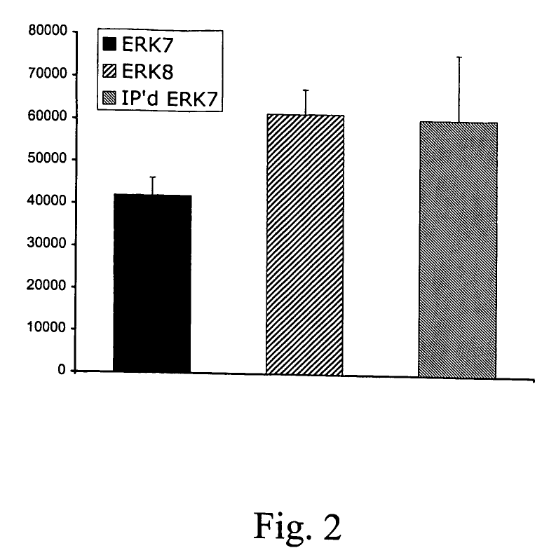

The kinase assays were characterized with respect to concentration of ERK7 and ERK8 required, time of incubation and concentration of ATP required. FIG. 1 demonstrates the K_{M} of Mn⁺⁺-ATP for bacterially expressed ERK7. The K_{M} is approximately 100 μM. Also shown in FIG. 1 is the high signal:background ratio that can be achieved by this assay. Kinase activity in the presence of 1 mM ATP of the recombinant ERK7 and ERK8 and immunoprecipitated ERK7 is depicted in FIG. 2. Thus, the assays are suitable for measuring activity of ERK7 and ERK8.

1. A method of diagnosing the presence of an estrogen responsive cancer in a patient, said method comprising the step of
measuring ERK8 levels in the cells of a biological sample obtained from said patient; and
determining if the ERK8 levels of the biological sample are significantly lower than those detected in non-cancerous cells, wherein significantly lower ERK8 levels indicates the presence of cancer cells in said patient.

2. The method of claim 1 wherein a decrease of greater than 20% in the ERK8 levels of the biological sample relative to the non-cancer cell levels indicates the presence of cancer cells in said patient.

3. The method of claim 2 wherein the estrogen responsive cancer to be diagnosed is selected from the group consisting of breast, ovarian and endometrial cancers.

4. The method of claim 3 wherein the estrogen responsive cancer to be diagnosed is breast cancer.

5. The method of claim 1 wherein the ERK8 level measured is the concentration of the ERK8 protein.

6. The method of claim 1 wherein the ERK8 level measured is the ERK8 kinase activity.

7. A method for diagnosing and determining the prognosis of a cancer patient, said method comprising the step of
determining ERK8 levels in a biopsy sample, wherein the severity of the decrease in ERK8 levels is correlated with more advanced stages of the cancer.

8. The method of claim 7 further comprising the step of determining estrogen receptor alpha (ER α) levels in the biopsy sample, wherein increased estrogen receptor alpha (ER α) levels (relative to healthy population levels) in combination with below normal ERK8 levels indicates a more advanced stage of cancer.

9. An antibody that specifically binds to the amino acid sequence of SEQ ID NO: 1.

10. The antibody of claim 9 wherein the antibody is labeled.

11. The antibody of claim 10, wherein the antibody is a monoclonal antibody.

12. The antibody of claim 11 wherein the antibody is releasably bound to a solid support.

13. A device for measuring ERK7/8 levels, said device comprising
a base provided with a capillary space, said capillary space having an interior surface and a first and second end;
a first port formed on an exterior surface of said base and in fluid communication with the first end of said capillary space;
a second port formed on an exterior surface of said base and in fluid communication with the second end of capillary space;
a reaction zone and a detection zone each formed on said interior surface of the capillary space, wherein said reaction zone comprises a labeled ERK7/8 antibody and the detection zone is located more proximal to the second port than the reaction zone, wherein fluid introduced into said first port will travel through the capillary space, contacting the reaction zone and then the detection zone as the fluid moves to the second port.

14. The device of claim 13 wherein said detection zone comprises a secondary antibody specific for said ERK7/8 antibody.

15. A method of monitoring the effectiveness of an anti-cancer agent for treating estrogen responsive cancers, said method comprising the steps of monitoring ERK8 levels in estrogen responsive cancer cells contacted with said agent.

16. The method of claim 15 wherein the cancer cells are contacted in vitro as a means of identifying new anti-cancer agents.

17. The method of claim 16 wherein the cancer cells are selected from various established tumor cell lines.

18. The method of claim 15 wherein the cancer cells are recovered from a patient and treated in vitro.

19. A method for identifying modulators of ERK7/8 activity, said method comprising the steps of
contacting an ERK7/8 polypeptide with a kinase substrate, and ATP, to form a control mixture;
contacting an ERK7/8 polypeptide with a kinase substrate, ATP, and a potential ERK7/8 activity modulator, to form a reaction mixture;
incubating the control and reaction mixtures under identical conditions for a predetermined length of time; and
comparing the amount of phosphorylated substrate produced in the reaction mixture to that produced in the control mixture, wherein a different concentrations of phosphorylated substrate produced by the control and reaction mixtures identifies a ERK7/8 activity modulator.

20. The method of claim 19 wherein the kinase substrate is a polypeptide comprising the sequence of SEQ ID NO: 6.


SEQUENCE LISTING (6 sequences)

SEQ ID NO: 1 - protein, 484 aa, Homo sapiens
MCTVVDPRIVRRYLLRRQLGQGAYGIVWKAVDRRTGEVVAIKKIFDAFRDKTDAQRTFREITLLQEFGDHPNIISLLDVIRAENDRDIYLVFEFMDTDLNAVIRKGGLLQDVHVRSIFYQLLRATRFLHSGHVVHRDQKPSNVLLDANCTVKLCDFGLARSLGDLPEGPEDQAVTEYVAEDLLALGSGCRASVLHQLGSRPRQTLDALLPPDTSPEALDLLRRLLVFAPDKRLSATQALQHPYVQRFHCPSDEWAREADVRPRAHEGVQLSVPEYRSRVYQMILECGGSSGTSREKGPEGVSPSQAHLHKPRADPQLPSRTPVQGPRPRPQSSPGHDPAEHESPRAAKNVPRQNSAPLLQTALLGNGERPPGAKEAPPLTLSLVKPSGRGAAPSLTSQAAAQVANQALIRGDWNRGGGVRVASVQQVPPRLPPEARPGRRMFSTSALQGAQGGARALLGGYSQAYGTVCHSALGHLPLLEGHHV

SEQ ID NO: 2 - protein, 547 aa, Rattus norvegicus
MCAAEVDRHVSQRYLIKRRLGKGAYGIVWKAMDRRTGEVVAIKKIFDAFRDQTDAQRTFREIMLLREFGGHPNIIRLLDVIPAKNDRDIYLVFESMDTDLNAVIQKGRLLEDIHKRCIFYQLLRATKFIHSGRVIHRDQKPANVLLDAACRVKLCDFGLARSLSDFPEGPGGQALTEYVATRWYRAPEVLLSSRWYTPGVDMWSLGCILGEMLRGQPLFPGTSTFHQLELILETIPLPSMEELQGLGSDYSALILQNLGSRPRQTLDALLPPDTPPEALDLLKRLLAFAPDKRLSAEQALQHPYVQRFHCPDREWTRGSDVRLPVHEGDQLSAPEYRNRLYQMILERRRNSRSPREEDLGVVASRAELRASQRQSLKPGVLPQVLAETPARKRGPKPQNGHGHDPEHVEVRRQSSDPLYQLPPPGSGERPPGATGEPPSAPSGVKTHVRAVAPSLTSQAAAQAANQPLIRSDPARGGGPRAVGARRVPSRLPREAPEPRPGRRMFGISVSQGAQGAARAALGGYSQAYGTVCRSALGRLPLLPGPRA

SEQ ID NO: 3 - protein, 549 aa, Mus musculus
MCAAEVDRHVAQRYLIKRRLGKGAYGIVWKAMDRRTGEVVAIKKIFDAFRDQIDAQRTFREIMLLKEFGGHPNIIRLLDVIPAKNDRDIYLVFESMDTDLNAVIQKGRLLKDIHKRCIFYQLLRATKFIHSGRVIHRDQKPANVLLDSACRVKLCDFGLARSLGDLPEGPGGQALTEYVATRWYRAPEVLLSSRWYTPGVDMWSLGCILGEMLRGQPLFPGTSTFHQLELILKTIPLPSMEELQDLGSDYSALILQNLGSRPQQTLDALLPPDTPPEALDLLKRLLAFAPDKRLSAEQALQHPYVQRFHCPDREWARESDVRLPVHEGDQLSAPEYRKRLYQIILEQSGNSRSPREEGLGVVASRAELRASPARTQSLKSGVLPQVPAETPARKRGPKPPRSPGHDPEHVEVRRQSSDPLFQLPPPGRGERPPGATGQPPSAPSGVKTQVRAMAPSLTSQAEAQAANQALIRSDPARGGGPRAVGARRVPSRLPREAPEPRPGRRMFGISVSQGAQGAARAALGGYSQAYGTVCRSALGRLPLLPGPRA

SEQ ID NO: 4 - protein, 16 aa, Rattus norvegicus
CRSALGRLPLLPGPRA

SEQ ID NO: 5 - protein, 7 aa, Rattus norvegicus
CQALTEY

SEQ ID NO: 6 - protein, 16 aa, Homo sapiens
KNIVTPRTPPPSQGKG